(19) United States
(12) Patent Application Publication
(10) Pub. No.: US 2025/0369941 A1
(43) Pub. Date: Dec. 4, 2025

(54) ARRANGEMENT WITH A GAS MEASURING DEVICE AND A CONTAINER AND METHOD USING SUCH AN ARRANGEMENT

(71) Applicant: Dräger Safety AG & Co. KGaA, Lübeck (DE)

(72) Inventors: Christian Wilhelm, Lübeck (DE); Hannes Sturm, Lübeck (DE); Florian Hansen, Lübeck (DE)

(21) Appl. No.: 19/218,761

(22) Filed: May 27, 2025

(30) Foreign Application Priority Data
May 30, 2024 (DE) 102024115154.4

(51) Int. Cl. G01N 33/00; G08B 21/16

(52) U.S. Cl. CPC G01N 33/0063; G08B 21/16

(57) ABSTRACT

The present disclosure relates to an arrangement and to a method which monitor a spatial area in which people are or may be present. A gas measuring device measures the concentration of a target gas in its detection region and generates a signal which contains information about the measured target gas concentration. A container accommodates an object in its interior, in particular for rescuing people. A closure mechanism closes the container in a closing state so that access to the object in the container is blocked. In a releasing state, the closure mechanism allows the object to be removed from the container. A signal-processing control unit receives and processes the signal from the gas measuring device and automatically decides whether the target gas concentration is too high. If the target gas concentration is too high, the control unit causes the closure mechanism to be transferred into the releasing state.

### CROSS-REFERENCE TO RELATED APPLICATIONS

This application claims the priority of German Patent Application No. 102024115154.4, filed on May 30, 2024, and titled “ARRANGEMENT WITH A GAS MEASURING DEVICE AND A CONTAINER AND METHOD USING SUCH AN ARRANGEMENT,” which is hereby incorporated by reference in its entirety for all nonlimiting purposes.

### SUMMARY

The present disclosure relates to an arrangement and a method which can monitor a spatial area in which people are or may be present for dangerous situations. In particular, the spatial area is in the interior of a building or a vehicle but can also be an area in the open air.

The object of the present disclosure is that of providing an arrangement and a method which allow for better protection of people in a spatial area than known arrangements, these people potentially being put at risk from too much harmful gas or too little vital gas.

The object is achieved by an arrangement and a method having the features described herein. Advantageous embodiments are specified in the depending claims. Insofar as they are reasonable, advantageous embodiments of the arrangement according to the present disclosure are also advantageous embodiments of the method according to the present disclosure, and vice versa.

The arrangement according to the present disclosure comprises at least one gas measuring device, optionally a plurality of gas measuring devices. The or each gas measuring device of the arrangement is configured to be installed in a respective stationary location and has a respective corresponding spatial detection region. In one embodiment, the gas measuring device can be connected to a stationary power supply network. In one embodiment, the gas measuring device comprises its own power supply unit so as to be independent of a stationary power supply network. These two embodiments can be combined with one another.

The or each gas measuring device is configured to measure the concentration of at least one respective target gas in its corresponding detection region. A gas whose concentration can be measured by the or at least one gas measuring device of the arrangement is hereinafter referred to as a “target gas”. In one embodiment, the gas measuring device can measure the concentration of one target gas; in another embodiment, it can measure the respective concentration of a plurality of target gases or the sum of target gas concentrations. In one embodiment, the gas measuring device can measure the respective concentration of a plurality of simultaneously present target gases; in another embodiment, the gas measuring device can be configured, for example using a switch, so that it depends on the configuration which respective concentration of a target gas the gas measuring device measures. It is possible that two gas measuring devices of the arrangement can measure the concentration of the same target gas.

The detection region of the gas measuring device or the respective detection regions of the gas measuring devices of the arrangement collectively cover(s) a spatial area in which people are or can be present and in which at least one target gas is or can be present. Two different gas measuring devices of the arrangement can have the same detection region or two different detection regions.

The or at least one target gas may be a gas which, in a sufficiently high concentration, is harmful to humans, in particular a flammable or toxic target gas. The or a target gas can also be a gas vital for humans, for example oxygen, or can be an anesthetic agent. In many cases, a person using any of his/her five senses is unable to determine that the target gas concentration is outside a specified value range. Therefore, often a gas measuring device is required to detect this dangerous situation.

Note: The formulation is used that a sensor is able to measure a physical variable, for example the concentration of a target gas. This formulation means that the sensor is able to directly measure the physical variable or at least another variable that correlates with the variable to be measured. The or a measured other variable or collectively the combination of the other measured variables are therefore an indicator for the physical variable to be measured. The measurement provides at least one value for the sought physical variable.

Furthermore, the or each gas measuring device can generate a signal. This signal comprises information about the or each target gas concentration measured by the generating gas measuring device.

Furthermore, the arrangement comprises a container. This container comprises a wall that encloses (surrounds) an interior, wherein preferably an aperture is inserted into the wall and a door or a lid or another closure is inserted in the wall and can close or release the variable. The container is configured to accommodate in its interior at least one object. Preferably, the or at least one of the objects accommodated in the interior is one of the following objects:
personal protective equipment or a personal protective equipment component such as a protective helmet or a self-rescuer with a generator for a breathable gas mixture and a breathing mask,
a tool with which a person can fight fire, such as a fire extinguisher or a fire blanket,
a tool with which a person can gain or enlarge an access to a room, for example a breaking tool or a ram,
a tool with which a person can seal a leak in a pipe,
a portable light source, in particular a flashlight,
a portable signaling unit such as an indicator lamp or a horn or a siren,
a portable gas measuring device which can measure the concentration of a target gas and preferably has its own output unit and its own power supply unit,
a portable smoke detector.

A controllable closure mechanism of the arrangement can selectively (either) be switched between a closing or a releasing state. Typically, an opening (aperture) is inserted in the wall of the container, and a movable closure, such as a door or a lid, closes or releases this opening depending on the position.

In the closing state, the closure mechanism closes the container in such a way that access to the interior and thus to the or every object located in the container is blocked. Typically, the closure mechanism closes the closure in the closing state and thereby prevents the closure from moving and thereby releasing the opening in the wall of the container. In the releasing state, the closure mechanism allows at least one, preferably every object to be removed from the interior of the container. In general, the closure can then be moved, and this allows access through the opening in the wall.

A respective data connection is permanently or at least temporarily established between a signal-processing control unit of the arrangement and the or each gas measuring device of the arrangement and preferably at least one, preferably each optionally additional sensor of the arrangement. The or each data connection can be established by means of a data line and/or wirelessly, i.e. by means of radio waves. Via this data connection, the control unit can receive from each gas measuring device of the arrangement the signal that this gas measuring device has generated and that contains information about a measured target gas concentration. Preferably, the control unit repeatedly queries the or each gas measuring device and thus checks whether the gas measuring device is still active.

The control unit can process the or each received signal. The control unit can decide the following: Does a target gas with a concentration outside a specified value range, in particular above a specified upper limit, occur in the detection region of the or at least one gas measuring device of the arrangement? A concentration outside the value range is also denoted as a harmful or inadmissible target gas concentration. The control unit makes this decision automatically and depending on at least one processed signal from a gas measuring device of the arrangement. If the target gas can be harmful to humans, usually an upper limit is specified as the value range, such that a concentration of this target gas below the upper limit is not dangerous for humans. Accordingly, usually a lower limit is specified as the value range for a vital gas, in particular for oxygen. This specified value range can differ from target gas to target gas.

As long as the or each target gas concentration is within the respective specified value range, in particular below the respective upper limit, and no other situation hazardous to humans is detected either, the closure mechanism is in the closing state. In the beginning of the method according to the present disclosure, the closure mechanism is in the closing state. Responsive to the event that the control unit has detected that a harmful (inadmissible) target gas concentration is present in the or at least one detection region, the control unit causes the following: The control unit controls the closure mechanism. This control causes the closure mechanism to be transferred into the releasing state. As a result, the container can be opened or is automatically opened, e.g. by a spring mechanism. It is now possible to remove the or at least one object from the container and use it.

The method according to the present disclosure comprises the corresponding steps.

If the concentration of the or a target gas is outside the specified value range, a situation may arise which is dangerous for a person if the person is in the monitored spatial area and therefore in the detection region of the or at least one of the gas measuring devices of the arrangement. The present disclosure makes it easier for a person to protect himself/herself from a harmful target gas concentration and/or to reduce or at least detect the emergence of a harmful target gas. Accordingly, the present disclosure makes it easier for a person to protect himself/herself from the effects of a too low concentration of a vital target gas. At least one suitable object is present in the container. The object can be taken (removed) from the container if the closure mechanism is in the releasing state.

It would be possible not to provide any container at all for the object or to implement the container in such a way that at least one object can be removed from the container at any time. However, in this case there would be the risk of the object being removed from the container without authorization and then the object would no longer be there if it is needed. This problem is known, for example, from publicly accessible lifebuoys (lifesavers). The closure mechanism according to the present disclosure completely prevents or at least reduces the risk of an object being removed from the container without authorization, specifically outside of an emergency situation, and then not being there in an emergency. In addition, the closure mechanism reduces the risk of an object being removed and used outside of an emergency situation and then put back again and then not being able to fulfill its life-saving function at all or unable to fulfill it sufficiently safely (reliably) in an emergency situation.

It is possible that the closure mechanism according to the present disclosure comprises:
a mechanical lock that can be opened with a matching mechanical key and/or
a chip card reader for a matching chip card and/or
an input unit for a password, and/or
a reader for a biometric feature of a human.

This embodiment allows an authorized person to use or check or replace an object in the container, including outside of an emergency situation and therefore in the absence of time pressure.

However, there is a serious risk of not finding at all a matching key or matching chip card in an emergency situation, or only finding it after a relatively long time search time. In an emergency situation, the password may not be available. There is also the risk that a person will panic in an emergency situation, and he/she will not be able to enter the correct password. A human knowing a password or from whom a biometric feature is stored may not be present and available in an emergency situation.

The control unit automatically decides whether or not at least one target gas is present with a concentration outside the respective specified value range. In response to a concentration outside the value range, the control unit controls the closure mechanism, and thereby causes the closure mechanism to be transferred into the releasing state and at least one object to be removable from the container. In particular owing to this feature, the present disclosure eliminates the need for a human to have to open the container using a key or a chip card or a password or a biometric feature. Rather, according to the present disclosure, the control unit controls the closure mechanism in the event of a harmful target gas concentration, and thereby causes the closure mechanism to be transferred into the releasing state. Now an object can be taken from the container. In an emergency situation, neither a mechanical key or a chip card or a password or a biometric feature are required.

The arrangement according to the present disclosure can be used in combination with an alarm unit, wherein this alarm unit issues an alarm in at least one form perceptible to a human if a target gas concentration outside of the value range has been detected. The arrangement can also be used in combination with a remote emergency center, wherein the emergency center receives and displays signals from the gas measuring devices and optionally signals from other sensors, and wherein, in the event of a harmful target gas concentration, a person in the emergency center remotely initiates at least one suitable rescue measure and in particular causes the container to be opened.

However, the present disclosure eliminates the need for a human at place or in an emergency center to trigger or perceive the output of an alarm and, in response to the alarm, to open the container or cause the container to be opened. Instead, the control unit does this automatically. This feature of the arrangement according to the present disclosure reduces the risk of a person in the danger zone being put at risk due to his/her own wrong decision or due to some other person making a wrong decision or not making at all a decision. In particular, the wrong decision can result from a person not perceiving an alarm. In addition, in many cases the features described herein save time, especially because a control unit often reacts faster than a human.

According to the present disclosure, the or each gas measuring device of the arrangement measures a respective target gas concentration. Preferably, the control unit automatically decides whether the or each measured target gas concentration is within or outside the respective value range. This feature eliminates the need to store a computer-evaluable identifier or characterization for the or each value range on the gas measuring device itself and change it if necessary. Instead, it is sufficient to store the respective identifier for the value ranges on the control unit. In order to decide whether an emergency situation occurs, the control unit can also apply a predefined evaluation rule, wherein this evaluation rule depends on at least one target gas concentration and optionally on another measured value, for example on a measured further target gas concentration or an ambient condition or on the occurrence of smoke. The evaluation rule can therefore depend on the signals of a plurality of sensors.

As already explained, the detection regions of the gas measuring devices of the arrangement collectively cover a spatial area to be monitored, in which spatial area people are present or at least can be present and in which spatial area an inadmissible target gas concentration may occur. Preferably, the container is located in this monitored spatial area or at least in the vicinity of the spatial area such that a person in the spatial area can quickly reach the container if necessary and take an object from the container. The control unit, however, can be located outside the spatial area. This makes it easier to protect the control unit from harmful influences that may occur in the spatial area. Furthermore, this embodiment makes it easier to ensure that the control unit is always supplied with electrical power and that a fault or failure of the control unit is quickly identified.

According to the present disclosure, the closure mechanism closes the container in the closing state. In the releasing state, an object can be taken (removed) from the container. In one embodiment, the closure mechanism comprises a locking body, a restoring (reset) element, and an actuator. The locking body is movable back and forth relative to the wall of the container and thus relative to a closure, in particular to a door, of the container between a closing and a releasing position. If the locking body is in the closing position, the closure mechanism is in the closing state. If the locking body is in the releasing position, the closure mechanism is in the releasing state.

The restoring element exerts a restoring force and endeavors to transfer the locking body into one of the two positions and thereby the closure mechanism into one of the two states and to hold it in this state by means of the restoring force. The actuator can be activated and deactivated by external control (actuation). The activated actuator endeavors to move the closure mechanism into the other one of the two states against the restoring force. The control unit can control the actuator. This control causes the actuator to be activated or deactivated. The restoring element is preferably configured as a passive component which does not require a supply of electrical or hydraulic or pneumatic energy and also does not need to be controlled for being activated.

It is possible for the restoring element to endeavor to move the locking body into the closing position and thereby keep the closure mechanism in the closing state. Preferably, however, the restoring element endeavors to move the locking body into the releasing position and thereby transfer the closure mechanism into the releasing state. The activated actuator holds the closure mechanism in the closing state against the restoring force. If the control unit has detected a harmful target gas concentration, the control unit will cause the actuator to be deactivated by means of an appropriate arrangement. By applying the restoring force, the restoring element then transfers the locking body into the releasing position.

A background to this preferred implementation is as follows: Typically, the actuator requires electrical power to overcome (surpass) the restoring force. Should the actuator or the electrical power supply fail, the actuator can no longer perform this action. In particular in the event of a failure of the power supply, a safe state is established if the closure mechanism is transferred into the releasing state. Sometimes, the power supply is intentionally switched off, especially in the event of a harmful target gas concentration, optionally in connection with a fire, or fails.

In one embodiment, a person can open a closure of the container, for example pivot a door, if the closure mechanism is in the releasing state. In an example implementation, the container comprises an opening mechanism. Preferably, the opening mechanism is also configured as a passive component and does not require any electrical or pneumatic or hydraulic energy or control. The opening mechanism endeavors to open the closure such that, if the closure mechanism is transferred into the releasing state, the closure springs open without any human action being required to open the closure. In one embodiment, the opening mechanism comprised a mechanical or pneumatic spring.

In one embodiment, the arrangement additionally comprises a smoke detector (smoke alarm generator). This smoke detector can detect smoke in its detection region. It is well known that smoke in a high concentration and fire are also harmful to humans, namely because humans may suffer from smoke poisoning or also from burns. If the smoke detector has detected smoke, the smoke detector generates a signal, wherein the signal includes information about the detection of smoke. This signal is transmitted to the control unit. In response to receiving this signal, the control unit causes the closure mechanism to be transferred to the releasing state, preferably independent from a measured target gas concentration.

As a result of this embodiment, in the event of a high target gas concentration or also in the event of a fire or smoke, the container is opened, and an object can be removed from the container. Optionally, the smoke detector also issues an alarm, preferably in an acoustic form. It is possible, but owing to this embodiment not necessary, for a person to perceive the alarm and then react correctly to the alarm.

In one embodiment, the arrangement comprises at least one signaling unit. In one embodiment, the or a signaling unit is arranged on the container, or at least one signaling unit is arranged in the vicinity of the container. In another embodiment, the or one signaling unit is a portable device and can be carried by a person. It is possible that the arrangement comprises at least one signaling unit on the container and additionally at least one portable signaling unit. The or every signaling unit can automatically be activated by external control. In response to having been activated, the signaling unit produces (generates) an output in at least one form perceptible to a human, in particular visually or acoustically. A human can perceive this output from outside the container. For example, the signaling unit includes an indicator lamp or a horn or a siren. It is possible that the arrangement comprises a first signaling unit for a visual output and a second signaling unit for an acoustic output.

As long as the or each target gas concentration is within the respective value range and the optional smoke detector has not detected any smoke, the signaling unit is not activated and does not produce an output or produces a standard output. According to the present disclosure, the control unit transfers the closure mechanism into the releasing state if a harmful target gas concentration is present or if the optional smoke detector has detected smoke. According to the embodiment with the signaling unit, in this situation the control unit additionally controls the signaling unit and thereby activates the signaling unit. The activated signaling unit produces the output described above. In one embodiment, after activation, the signaling unit produces the output in a different manner than before activation.

In many cases, the signaling unit makes it easier for a person to find the container quickly, in particular if the signaling unit is mounted on the container. Since the signaling unit does not always produce the output in the same manner but only, or in a different manner, after being activated by control, the output of the signaling unit is better recognized than if the signaling unit were to always produce the same output.

In one embodiment, the arrangement additionally comprises a display unit that can be controlled externally. In response to a control signal, the display unit outputs at least one piece of information in at least one form perceptible by a human, in particular visually, from outside the container. In particular, this information includes one of the following pieces of information:
an acoustically and/or visually perceptible alarm indicating that a target gas concentration that is too high or too low and/or optionally that smoke has arisen,
information about a route to the container with the object and the closure mechanism, for example a direction indicator,
information about a spatial position of the container with the object and the closure mechanism, for example according to predetermined coding for areas in a building.

Optionally, the controlled display unit can also issue an indicator of how serious and/or dangerous the current situation is and/or a request to leave the spatial area now.

According to the present disclosure, the control unit can control the closure mechanism. In one embodiment, the control unit can additionally control the display unit just described. The control unit control the display unit if a harmful target gas concentration has been measured and/or if the optional smoke detector has detected smoke.

The display unit makes it easier for a person to quickly find the container with the closure mechanism, especially if the person is unfamiliar with a building in which the container is positioned.

### BRIEF DESCRIPTION OF THE DRAWINGS

In the following, the disclosure is described on the basis of an exemplary embodiment. In the drawings,

FIG. 1 shows the components of an example arrangement; and

FIG. 2 shows an embodiment of the closure mechanism in an example arrangement.

### DETAILED DESCRIPTION

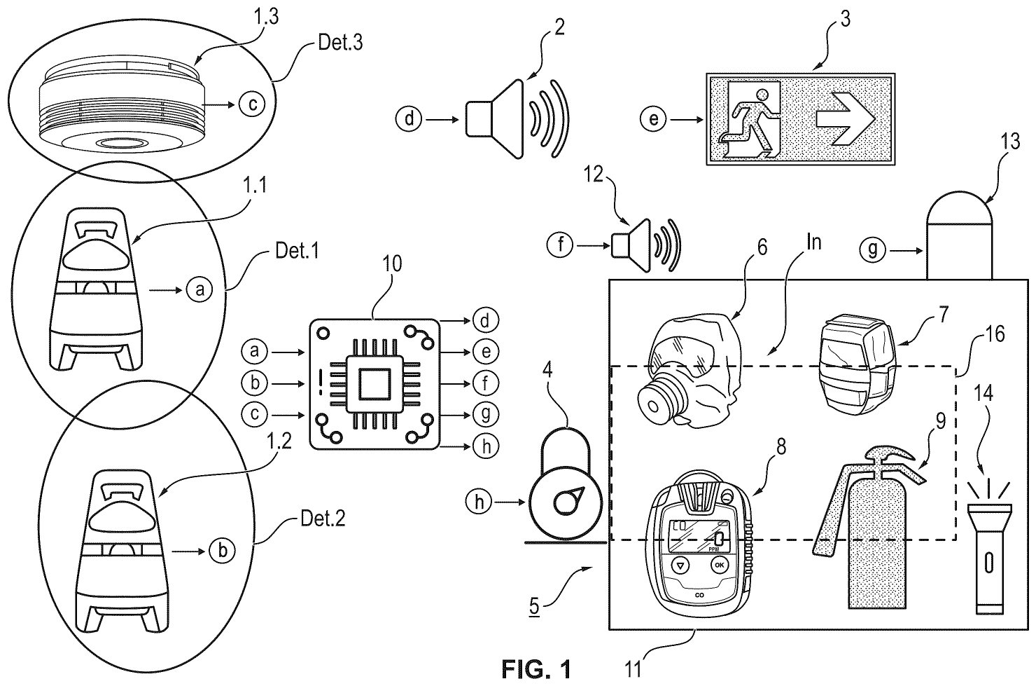

FIG. 1 shows an example embodiment of the arrangement according to the present disclosure. The components of this arrangement will be explained below.

The arrangement is used in a spatial area in which people (humans) are, or at least may be, present. Examples of such a spatial area are public places, buildings, vehicles, storage facilities, or industrial plants.

In this spatial area, at least one gas may arise which, at a sufficiently high concentration, is flammable and/or toxic and/or in some other way harmful to humans. Such a harmful gas will hereinafter be referred to as a “target gas”.

Two stationary gas measuring devices 1.1, 1.2 of the arrangement are installed at two locations spaced apart from one another. The term “stationary device” specifies that the device is configured to be installed and/or mounted in one location and afterwards used there. The stationary device 1.1, 1.2 can generate a signal. The signal contains information about a measurement result of the gas measuring device 1.1, 1.2 and preferably a unique identifier of the gas measuring device 1.1, 1.2 and/or a timestamp. The stationary device 1.1, 1.2 comprises a communication unit, can generate a signal, and can transmit the generated signal to a spatially remote receiver by means of said communication unit. In particular, the communication unit can transmit the signal by wire and/or radio waves. The stationary gas measuring device 1.1, 1.2 does not necessarily comprise its own output unit which outputs measurement results or alarms in at least one form perceptible to a human. Optionally, the stationary gas measuring device 1.1, 1.2 comprises at least one status lamp that indicates an internal status of the device.

Each gas measuring device 1.1, 1.2 has a (schematically shown) detection region Det.1, Det.2. Each gas measuring device 1.1, 1.2 can measure the respective concentration of at least one target gas in its respective detection region Det.1, Det.2. The detection regions Det. 1, Det.2 collectively cover at least the part of the spatial area in which people are or can be present and in which there can be a target gas concentration harmful to humans.

Each gas measuring device 1.1, 1.2 can generate a signal. This signal contains information about the target gas concentration that this gas measuring device 1.1, 1.2 has measured. In one embodiment, the signal contains information as to whether the measured target gas concentration is above a predetermined upper limit or not, but does not necessarily contain the measured target gas concentration itself. Each upper limit is specified in such a way that no danger to humans can occur if the target gas concentration is less than or equal to the upper limit. In another embodiment, the signal comprises an identifier (characterization) for the measured target gas concentration. A control unit 10 (described below) can determine whether this target gas concentration is too high or not. These embodiments can be combined with one another. Optionally, the signal also includes information about the geoposition and/or a unique identifier of the gas measuring device 1.1, 1.2 and/or a time stamp for a measured value.

It is possible that all the gas measuring devices 1.1, 1.2 are configured to detect the same target gas. It is also possible that the gas measuring devices 1.1, 1.2 in their entirety are configured to detect at least two different target gases.

A smoke detector 1.3 can detect an indication of the occurrence of smoke. It is known that smoke is often an indication of fire. The smoke detector 1.3 also has a detection region Det.3. If smoke occurs in this detection region Det.3, the smoke detector 1.3 will detect this smoke. In this case, the smoke detector 1.3 is configured to generate a signal with corresponding information.

In the shown exemplary embodiment, two gas measuring devices 1.1, 1.2 and one smoke detector 1.3 are used. Of course, a different number of gas measuring devices and/or smoke detectors is also possible.

Preferably, each gas measuring device 1.1, 1.2 and the smoke detector 1.3 comprise a respective measuring chamber, and the measuring chamber can accommodate a gas sample from the respective detection region Det. 1, Det.2, Det.3. Different detection principles or measuring principles as to how a gas measuring device 1.1, 1.2 can detect a target gas in a gas sample and/or measure the concentration of a target gas in the gas sample are possible. The two gas measuring devices 1.1, 1.2 can apply the same measuring principle or different measuring principles. For example, a radiation source emits electromagnetic radiation or sound into the measuring chamber, a target gas in the measuring chamber attenuates the radiation or sound, and a detector measures the intensity of incident radiation or incident sound. The measured intensity correlates with the target gas concentration. The smoke detector 1.3 can also apply a corresponding measuring principle to detect smoke in a gas sample.

A signal-processing control unit 10 receives a respective signal from each of the two gas measuring devices 1.1, 1.2 and the smoke detector 1.3. The signal from a gas measuring device 1.1, 1.2 contains information about the measured target gas concentration, the signal from the smoke detector 1.3 contains information about the detection or absence and optionally intensity of smoke. In one embodiment, the signal from a gas measuring device 1.1, 1.2 contains the information about a measured target gas concentration. The control unit 10 decides whether this target gas concentration is above the specified upper limit. In another embodiment, the gas measuring device 1.1, 1.2 itself decides whether the target gas concentration is above the upper limit, and the signal contains information about this result.

The term “alarm situation” is used below. An alarm situation has occurred if a target gas concentration is above the specified upper limit and/or if smoke has been detected. An alarm situation has therefore also occurred if the smoke detector 1.3 has detected smoke.

The control unit 10 can control an output unit 2. Depending on the control, this output unit 2 can output messages that can be perceived by a human, in particular acoustically. In one embodiment, the control unit 10 automatically generates a voice message from predefined and stored voice message modules in the event of an alarm situation. The generated voice message indicates, on the one hand, the alarm situation that has occurred and, on the other hand, information about measures that must now be taken.

The output voice message characterizes the alarm situation, for example, by means of the following information:
Which target gas has triggered the alarm situation? Or did the detection of smoke trigger the alarm situation?
Where was this target gas or smoke detected? This information is derived from which one of the gas measuring devices 1.1, 1.2 detected an excessive target gas concentration and/or which smoke detector 1.3 detected smoke.
optionally, an identifier for the target gas concentration or the strength of the smoke.

The output information about measures to currently be taken may include, for example, the following actions:
The monitored spatial area must be vacated.
The location of a gathering point to which people in the spatial area should proceed to is described.
A list of whether and, if so, which personal protective equipment must now be worn.
Optionally, a description is given as to how to put on personal protective equipment.
Optionally, a description is given as to which tool is now to be used.
The route to a container with protective equipment and tools is described.
   This container is described below.

In one embodiment, the control unit 10 configures the information about

measures depending on which device has transmitted a signal with an alarm situation. For example, if a gas measuring device 1.1, 1.2 has detected a toxic target gas at a high concentration, the information about the measures includes the instruction to wear gas masks and, optionally, the instruction to carry a portable gas measuring device. If a smoke detector 1.3 has detected smoke, the information includes, for example, the instruction to use a fire extinguisher or fire blanket.

Furthermore, the control unit 10 controls a display unit 3 in an alarm situation. This display unit 3 indicates in a form perceptible to a human, in particular visually, where to go in order to leave a now dangerous spatial area. In the shown example, the display unit 3 visually indicates a direction to a safe zone, for example to a gathering point (collection point). In one embodiment, the display unit 3 is in a rest state provided it is not controlled and thereby activated. The step of the control unit 10 to control the display unit 3 causes the display unit 3 to be transferred into a display state. In one embodiment, the control causes the display unit 3 to be illuminated and/or to flash.

The aforementioned container with protective equipment and tools will be described in more detail below. The closable container 5 comprises a wall 11 which encloses an interior In. The container 5 is configured, for example, as a cabinet or cupboard. An opening and a closure in the form of a door 16 are inserted in the wall 11. The door 16 can be rotated relative to the wall 11 about a vertical axis of rotation and can thus open and close. The closed door closes the opening in the wall 11. In the exemplary embodiment, an acoustic signaling unit 12, for example a horn, and a visual signaling unit 13, for example a flashing indicator lamp 13, are arranged on the outside of the wall 11.

The interior In is configured to accommodate a plurality of objects. There can be a plurality of shelves in the interior In. These objects can be used to escape from a region with a high concentration of target gas and/or to combat the emergence of target gas or the spread of a fire. FIG. 1 shows, by way of example, the following objects that are currently in the interior In:
an escape hood 6 which a user can place in front of his/her face and which comprises a filter for particles,
a self-rescuer 7 in a separate container, wherein the self-rescuer 7 has a face mask and an oxygen generator and is configured to generate a gas mixture comprising oxygen and to supply it to the face mask,
a portable gas measuring device 8 which a person can carry with him/her,
a fire extinguisher 9, and
a flashlight 14.

Other objects that may be in the container 5 are, for example,
a protective helmet,
a gas mask with a filter unit,
a ladder,
tools such as an axe and/or a hammer and/or a ram, and
an acoustic warning device, such as a portable horn or siren.

A closure mechanism 4 can either close the door 16 and thus the container 5 or allow access to the objects 6, 7, 8, 9, 14 in the container 5. If and while the closure mechanism 4 is in a closing state, the door 16 is closed and locked, and access to the objects 6, 7, 8, 9, 14 in the container 5 is prevented. The closure mechanism 4 is therefore either in a locking or in a releasing state. In the exemplary embodiment, the container 5 comprises the door 16. In the closing state, the closure mechanism 4 closes this door 16. If and while the closure mechanism 4 is in the releasing state, the door 16 can be opened or is opened automatically.

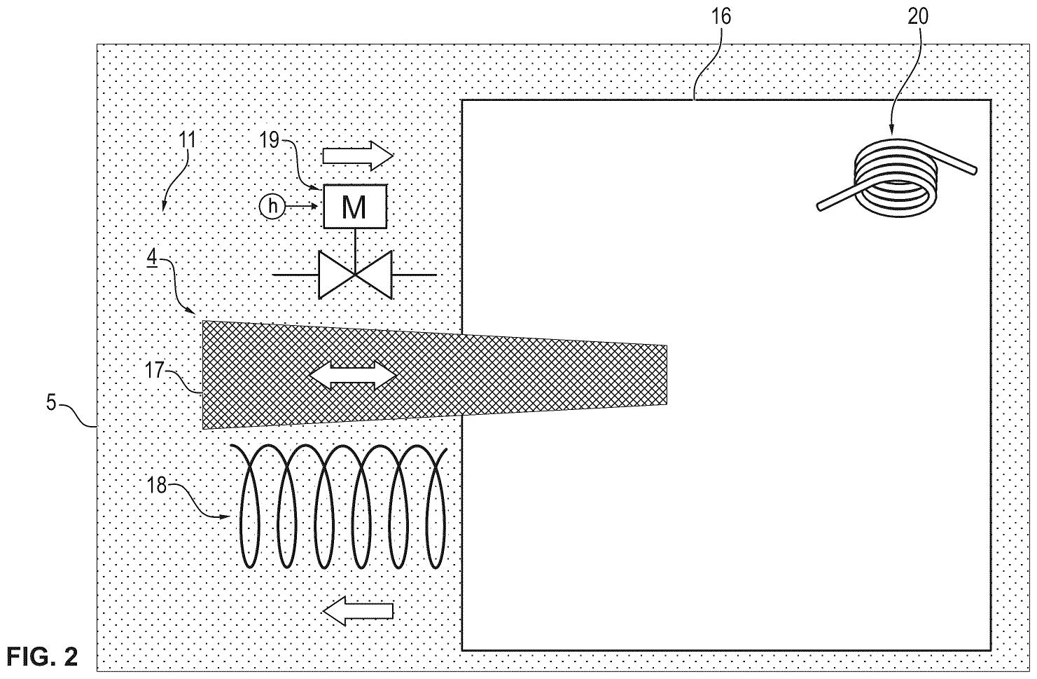

FIG. 2 shows an example embodiment of the closure mechanism 4. In this embodiment, a locking body 17 of the closure mechanism 4 can be moved back and forth relative to the wall 11 and thus to the door 16 between a closing position and a releasing position; in the exemplary embodiment, back and forth horizontally. In the closing position, the locking body 17 locks the door 16. The closure mechanism 4 is then in the closing state. FIG. 2 shows this closing position. In the releasing position, the locking body 17 allows the door 16 to be opened. The closure mechanism 4 is then in the releasing state.

In the shown implementation, the closure mechanism 4 further comprises a passive restoring element, for example a mechanical or pneumatic spring, which is supported on the wall 11, and an actuator 19. The term “passive” specifies that the restoring element 18 does not require a supply of electrical, pneumatic, and/or hydraulic energy, and also does not require external control. The restoring element 18 permanently exerts a restoring force—to the left in the example in FIG. 2. The restoring element 18 endeavors to move the locking body 17 into one position and to hold it in this position. The actuator 19 can be activated and deactivated by corresponding external control. The control unit 10 can control and thereby activate the actuator 19. The activated actuator 19 endeavors to move the locking body 17 against the restoring force of the restoring element 18 into the other position—to the right in the example in FIG. 2. If the actuator 19 is deactivated, only the restoring element 18 acts on the locking body 17.

In one implementation, the restoring element 18 endeavors to move the locking body 17 into the closing position. The activated actuator 19 moves the locking body 17 into the releasing position. FIG. 2 describes the opposite implementation. In this example, the restoring element 18 comprises a tension spring and endeavors to move the locking body 17 into the releasing position. The activated actuator 19 endeavors to move the locking body 17 into the closing position.

In one embodiment, a passive opening element, for example a torsion spring 20, endeavors to open the door 16 such that the door 16 springs open and keep it in an open state. This embodiment achieves the following: As soon as the closure mechanism 4 is transferred into the releasing state and the door 16 is no longer locked, the opening element 20 opens the door 16 without any human action being required for this.

The closure mechanism 4 can be externally controlled. Preferably, the closure mechanism 4 is in the locking state provided it is not controlled. Control causes the closure mechanism 4 to be transferred into the releasing state. In the example in FIG. 2, control of the actuator 19 causes the actuator 19 to be deactivated and thereby the restoring element 18 to move the locking body 17 into the releasing position.

FIG. 2 shows the following implementation: As long as no impermissible target gas concentration and no other alarm situation is detected, the actuator 19 is activated and holds the locking body 17 in the closing position. If the control unit 10 has detected an inadmissible target gas concentration or another alarm situation and access to objects in the container 4 is therefore necessary, the control unit 10 controls the actuator 19 and causes it to be activated. In response to this, the actuator 19 is deactivated and the restoring element 18 moves the locking body 17 into the releasing position.

As already explained, the control unit 10 detects an alarm situation, in particular if one of the following events has occurred:
A gas measuring device 1.1, 1.2 has measured a target gas concentration above the upper limit, and/or
The smoke detector 1.3 has detected smoke.

If the control unit 10 has detected an alarm situation, the control unit 10 triggers the following steps:
The control unit 10 controls the closure mechanism 4 and thereby causes the closure mechanism 4 to be transferred into the releasing state—in one embodiment by the restoring element 18 and in another embodiment against the restoring force of the restoring element 18;
The door 16 springs up or is opened manually. The objects 6, 7, 8, 9, 14 can then be removed from the container 5;
The control unit 10 controls the acoustic signaling unit 12 and causes it to emit a sound;
The control unit 10 controls the optical signaling unit 13 and causes it to issue a visually perceptible message.

The activated signaling units 12 and 13 make it easier for a user to find the cabinet 5, even in an alarm situation.

LIST OF REFERENCE SIGNS

| | |
|---|---|
| 1.1, 1.2 | stationary gas measuring device, measures the concentration of |
| | a harmful target gas in its respective detection region Det.1, |
| | Det.2 |
| 1.3 | smoke detector, has the detection region Det.3 |
| 2 | acoustic output unit |
| 3 | optical escape route display |
| 4 | controllable closure mechanism for the cabinet 5, comprises the |
| | locking body 17, the restoring element 18, and the actuator 19 |
| 5 | cabinet, accommodates the escape hood 6, the self-rescuer 7, |
| | the gas measuring device 8, the fire extinguisher 9, and the |
| | flashlight 14, closable by the closure mechanism 4, acts as the |
| | container |
| 6 | escape hood in the cabinet 5, comprises a particle filter and a |
| | face mask |
| 7 | self-rescuer in the cabinet 5, comprises an oxygen generator and |
| | an escape hood in a container |
| 8 | portable gas measuring device in the cabinet 5 |
| 9 | fire extinguisher in the cabinet 5 |
| 10 | signal-processing control unit, receives a respective signal from |
| | each stationary gas measuring device 1.1, 1.2 and from the |
| | smoke detector 1.3, controls and activates the loudspeakers 2, |
| | the escape route display 3, the closure mechanism 4, and the |
| | signaling units 12, 13 |
| 11 | wall of the container 5, carries the door 16 as well as the units |
| | 12 and 13 |
| 12 | acoustic signaling unit |
| 13 | visual reporting unit |
| 14 | flashlight in the closet 5 |
| 16 | door in the wall 11 |
| 17 | locking body, movable back and forth between a closing and a |
| | releasing position |
| 18 | restoring element in the form of a tension spring, endeavoring to |
| | move the locking body 17 into the releasing position |
| 19 | controllable actuator, endeavoring to move the locking body 17 |
| | into the closing position |
| 20 | opening mechanism for the door 16, comprises a torsion spring |
| Det.1, | detection region of the gas measuring device 1.1, 1.2 |
| Det.2 | |
| Det.3 | detection region of the smoke detector 1.3 |
| In | interior of the container 5, enclosed by the wall 11 |

1.-8. (canceled)

9. An arrangement comprising:
a gas measuring device, wherein the gas measuring device of the arrangement corresponds to a detection region;
a container with a wall surrounding an interior of the container;
a closure mechanism for the container; and
a signal-processing control unit,
wherein the gas measuring device is configured to:
be installed at a stationary location;
measure a concentration of a target gas in the corresponding detection region; and
generate a signal comprising information of the measured concentration,
wherein the container is configured to accommodate an object within the interior,
wherein the closure mechanism is configured to be transferred between a closing state and a releasing state,
wherein the closure mechanism:
based on being in the closing state, closes the container in such a way that access to the object accommodated in the container is blocked, and
based on being in the releasing state, allows the object to be removed from the container,
wherein the control unit is configured to:
receive, from the gas measuring device, the generated signal; and
to determine, based on the received signal, whether the concentration measured in the detection region of the gas measuring device is outside a value range specified for the target gas, and
wherein the control unit is further configured to:
cause the closure mechanism to be transferred into the releasing state as a reaction responsive to the event that the target gas has a measured concentration outside the specified value range.

10. The arrangement of claim 9, further comprising:
a smoke detector corresponding to a smoke detector detection region,
wherein the smoke detector is configured to:
detect whether smoke is present in the smoke detector detection region; and
generate a smoke detection signal comprising information of the presence of smoke,
wherein the control unit is configured to:
receive the smoke detection signal from the smoke detector; and
cause the closure mechanism to be transferred into the releasing state in response to receiving the smoke detection signal.

11. The arrangement of claim 9, further comprising:
a signaling unit, wherein the signaling unit is:
arranged on the container, or
configured to be carried by a human,
wherein the signaling unit is configured to:
be activated; and
in response to being activated, produce an output perceptible by a human from outside the container, and
wherein the control unit is configured to:
activate the signaling unit based on the detection of the measured concentration of the target gas being outside the specified value range.

12. The arrangement of claim 9, wherein the object comprises at least one of the following:
a component of personal protective equipment,
a source for breathable air,
a tool for fighting a fire,
a tool for gaining or enlarging access to a room,
a tool for sealing a leak,
a portable light source, and
a portable gas measuring device.

13. The arrangement of claim 9, further comprising:
a controllable display unit,
wherein the controllable display unit is configured to output, in response to a corresponding control, a piece of information in at least one form perceptible by a human from outside the container,
wherein the piece of information comprises one or more of:
a warning;
a direction indication; and
an identifier for a spatial position, and
wherein the control unit is configured to control the display unit to cause the piece of information to be output if the measured concentration of the target gas is outside the specified value range.

14. The arrangement of claim 9, wherein:
the container comprises a closure; and
the closure mechanism comprises:
a locking body;
a restoring element; and
an actuator configured to be activated and deactivated,
wherein the closure is inserted in the wall of the container and configured to be opened and closed,
wherein the closure, when opened, allows the object to be removed from the container,
wherein the locking body is movable back and forth relative to the closure between:
a closing position in which the locking body locks the closure; and
a releasing position allowing the closure to be opened,
wherein the restoring element is configured to:
exert a restoring force; and
based on exerting the restoring force, move the locking body toward one position of two positions, wherein the one position comprises:
the closing position, or
the releasing position,
wherein the actuator, based on being activated, is configured to move the locking body against the restoring force toward a second position of the two positions, and
wherein the control unit is configured to control the actuator.

15. The arrangement of claim 14:
wherein the restoring force exerted by the restoring element moves the locking body toward the releasing position, and
wherein the actuator, when activated, is configured to move the locking body against the restoring force toward the closing position, and
wherein the control unit is configured to:
deactivate the actuator, and
cause, based on deactivating the actuator, the locking body to be moved into the releasing position by the restoring force.

16. The arrangement of claim 9, wherein the gas measuring device comprises at least two gas measuring devices comprising:
a first gas measuring device corresponding to the detection region; and
a second gas measuring device corresponding to a second detection region and being configured to measure a concentration of a target gas in the corresponding second detection region.

17. The arrangement of claim 16, wherein at least a portion of the detection region overlaps with at least a portion of the second detection region.

18. The arrangement of claim 9, wherein the gas measuring device is communicatively coupled to the closure mechanism of the container.

19. A method for monitoring a spatial area:
wherein the method is carried out using an arrangement comprising:
a gas measuring device;
a container with a wall surrounding an interior of the container;
a closure mechanism for the container; and
a signal-processing control unit;
wherein the interior of the container accommodates an object,
wherein the gas measuring device of the arrangement:
is installed at a stationary location; and
corresponds to a detection region,
wherein initially the closure mechanism is in a closing state in which the closure mechanism closes the container in such a way that access to the object accommodated in the container is blocked,
wherein the method comprises the automatically performed steps in which:
the gas measuring device:
repeatedly measures a concentration of a target gas in the corresponding detection region; and
generates a signal comprising information of the measured concentration;
the control unit:
receives the signal from the gas measuring device; and
automatically determines, based on the received signal, whether the concentration measured in the detection region of the gas measuring device is outside a value range specified for the target gas; and
the control unit further:
causes the closure mechanism to be transferred from the closing state into a releasing state as a reaction responsive to the event that the target gas has a measured concentration outside the specified value range,
wherein the closure mechanism allows, in the releasing state, the object to be removed from the container.

20. The method of claim 19:
wherein the arrangement further comprises a smoke detector corresponding to a smoke detector detection region,
the method further comprising:
with the smoke detector, detecting whether smoke is present in the smoke detector detection region;
with the smoke detector, generating a smoke detector signal comprising information of the presence of smoke;
receiving, by the control unit, the smoke detector signal from the smoke detector; and
causing the closure mechanism to be transferred into the releasing state in response to receiving the smoke detector signal.

21. The method of claim 19:
wherein the arrangement further comprises a signaling unit, wherein the signaling unit is:
arranged on the container, or
carried by a human,
the method further comprising:
the control unit activating the signaling unit based on detection of the measured concentration of the target gas being outside the specified value range; and
as a response to being activated, producing with the signaling unit an output perceptible by a human from outside the container.

22. The method of claim 19, wherein the object comprises at least one of the following:
a component of personal protective equipment,
a source for breathable air,
a tool for fighting a fire,
a tool for gaining or enlarging access to a room,
a tool for sealing a leak,
a portable light source, and
a portable gas measuring device.

23. The method of claim 19:
wherein the arrangement further comprises a controllable display unit, wherein the controllable display unit is configured to output, in response to a corresponding control, a piece of information in at least one form perceptible by a human,
wherein the at least one piece of information comprises one or more of:
a warning;
a direction indication; and
an identifier for a spatial position, and
the method further comprising:
controlling, by the control unit, the display unit to cause the piece of information to be output if the measured concentration of the target gas is outside the specified value range.

24. The method of claim 19:
wherein the container comprises a closure, and
wherein the closure mechanism comprises:
a locking body;
a restoring element;
an actuator configured to be activated and deactivated,
wherein the closure is inserted in the wall of the container and configured to be opened and closed,
wherein the closure, when opened, allows the object to be removed from the container,
wherein the locking body is movable back and forth relative to the closure between:
a closing position in which the locking body locks the closure; and
a releasing position allowing the closure to be opened,
wherein initially the locking body is in the closing position, and
wherein the method further comprises:
exerting, by the restoring element, a restoring force;
based on exerting the restoring force, moving the locking body toward one position of two positions, wherein the one position comprises:
the closing position, or
the releasing position; and
based on activating the actuator, moving the locking body against the restoring force toward a second position of the two positions, and
wherein the control unit is configured to control the actuator.

25. The method of claim 24:
wherein the restoring force exerted by the restoring element moves the locking body toward the releasing position, and
wherein the actuator, when activated, moves the locking body against the restoring force toward the closing position, and
wherein the method further comprises:
the control unit deactivating the actuator as a reaction responsive to the event that the target gas has a measured concentration outside the specified value range, and
based on deactivating the actuator, the locking body moving into the releasing position.

26. The method of claim 19, wherein the gas measuring device comprises at least two gas measuring devices comprising:
a first gas measuring device corresponding to the detection region; and
a second gas measuring device corresponding to a second detection region and being configured to measure a concentration of a target gas in the corresponding second detection region.

27. The method of claim 26, wherein at least a portion of the detection region overlaps with at least a portion of the second detection region.

28. The method of claim 19, wherein the gas measuring device is communicatively coupled to the closure mechanism of the container.
